(19) United States
(12) Patent Application Publication
(10) Pub. No.: US 2009/0017139 A1
(43) Pub. Date: Jan. 15, 2009

(54) Iodine therapy dosing for treating medical conditions

(76) Inventor: Jack Kessler, Southborough, MA (US)

Correspondence Address: Baker & Hostetler LLP;Attn: Jim Coffman, 45 Rockefeller Plaza, New York, NY, 10111, US

(21) Appl. No.: 11/827,609

(22) Filed: Jul. 12, 2007

(51) Int. Cl. A61K 33/36; A61P 15/00

(52) U.S. Cl. 424/667

(57) ABSTRACT

A method for therapeutically treating medical conditions requiring chronic therapy such as fibrocystic breast disease, endometriosis, ovarian cysts, uterine fibroids and including the treatment of women considered to be at risk to breast and/or ovarian cancer without causing an overt thyroid disease by first administering to a patient a daily loading dose of molecular iodine (I₂) for a time period not to exceed six months and preferably 2 to 4 months at a concentration level sufficient to remediate symptoms associated with the medical condition followed by daily dosing with a maintenance dose of I₂ after treatment of the loading dose is completed with the concentration of the maintenance dose being substantially less than the concentration of the loading dose.

### FIELD OF THE INVENTION

The present invention relates to the treatment of human medical conditions requiring chronic therapy comprising the administration of molecular iodine (I₂) over a controlled time period using a specified first daily dosage of molecular iodine as a loading dose for a first interval of time immediately followed by a second daily dosage of molecular iodine as a maintenance dose over a second interval of time with the concentration of the second maintenance dosage being substantially less than the concentration of the first loading dose and preferably no greater than about 50-60% of the loading dose. More particularly the present invention relates to the treatment of human medical conditions requiring chronic therapy including, but not limited to, fibrocystic breast disease, endometriosis, ovarian cysts, uterine fibroids and breast and ovarian cancer prevention wherein said prevention is based at least in part upon reduction of estrogen receptors in the tissue of interest, e.g. breast, endometrium. More specifically, the daily loading dose of I₂ should be administered for up to 6 months and at an amount sufficient to remediate the symptoms associated with the medical condition immediately followed by a lower daily maintenance dose of I₂ to (1) preserve the clinical benefit and (2) minimize subclinical and clinical thyroid related findings.

### BACKGROUND OF THE INVENTION

The medical conditions targeted for treatment by the method of this application, although not well understood, are believed to arise from a subtle hormonal imbalance. The symptoms associated with these conditions are chronic and consequently patient compliance is an important consideration. Compliance with a therapeutic regimen for a chronic condition is improved if patients perceive a benefit in the earliest possible timeframe; likewise, the absence of unwanted side effects is an important consideration in patient compliance.

There is a substantial body of non-clinical and clinical observations suggesting that iodine intake impacts mammary health. When female rats are deprived of iodine they develop structural alterations in their mama: epithelial hyperplasia associated with mammary ducts and acinar cells, cyst formation and an increase in interacinar fibrosis (Eskin et al. 1995). This effect was found to be reversed by daily oral administration of iodine at daily levels above the amount known to be required to satisfy synthesis of thyroid hormones.

The conditions intended for treatment by the method of the present invention require chronic therapy. Chronic daily oral administration of an iodine therapeutic increases the focus on thyroid related adverse events or changes in the status of the thyroid as determined by thyroid function tests. The controlled clinical studies of this application demonstrate for the first time that the daily dose of I₂ is a safety consideration as dose is related to the incidence of subclinical hypothyroidism. Accordingly, it is beneficial to determine the safest effective dosage regimen of iodine to administer orally. It is important to note that the intended treatment population are those individuals known to be able to tolerate large daily doses of iodine without effect. Iodine is the oldest antithyroid drug known and consequently clinicians understand the clinical parameters that define the group of patients that tolerate iodine; if patients are euthyroid with no prior history of thyroid disease they can almost always tolerate high daily levels of iodine (Iodide was the first form of iodine used experimentally to reverse symptoms of mastalgia and fibrosis but I₂ has been shown to be more effective than iodide in remediation of these symptoms. In addition, I₂ has been determined to be less thyrotoxic than iodide in a number of controlled studies. These and other observations have adduced an interest in the use of I₂ to treat the following indications: fibrocystic breast disease, endometriosis, ovarian cysts, uterine fibroids and cancer prevention wherein said prevention is based at least in part upon reduction of estrogen receptors in the tissue of interest.

The relative oral toxicity of iodide as compared to I₂ has been examined by a number of researchers in a range of model systems. I₂ has repeatedly been shown to be less thyrotoxic than iodide. Acute exposure of iodide in fasted rats resulted in rapid distribution of iodine into the thyroid. Thyroid uptake of iodine atoms from I₂ was depressed as compared to iodide; and retention of iodine in thyroid at 72 hours was about 30% less (Thrall 1990).

Further, when iodine was administered as I₂ as compared to iodide, a statistically significant different association of radioactive iodine was observed in whole blood, blood cells, lipids, albumin, globulin and serum water. It was determined that a larger percentage of the iodine administered as I₂ was retained in the stomach contents and covalently bound to a lipid material (Thrall et al. 1992). The percentage of iodine retained in thyroid 72 hours after I₂ administration was significantly less than the amount observed from iodide (Thrall 1990).

I₂ in drinking water was less effective than iodide in suppressing thyroid uptake of a challenge dose of ¹²⁵iodide in dosing studies conducted for 100 days. Significant increases in thyroid size were observed in male rates treated with iodide but not with I₂ (Sherer et al. 1991).

Subsequent studies (Garcia-Solis et al. 2005) examined the influence of perchlorate on iodine incorporation into the thyroid from orally administered I₂ or iodide. The administration of I₂ resulted in half as much iodine uptake in thyroid as iodide; perchlorate (6 mg i.p.-2 hr predosing) reduced thyroidal uptake from iodide by more than 90% as compared to only 40% with I₂.

All studies that have used a valid I₂ dosage form have concluded that I₂ and iodide have distinct toxicological properties. The literature must, however, be read with care as some studies that purport to examine I₂ suffer from inappropriate handling of I₂ (Robison et al. 1998). The study by Robinson et al. does not test I₂ since this molecule reacts with water at a neutral pH and therefore cannot be stored in an aqueous environment for periods of time beyond 2-8 hours.

The non-clinical observations combined with human experimentation led to clinical use of I₂ to treat a number of human conditions. Clinical results from over 1,300 women exposed for up to five years to a regimen of daily I₂ therapy for treatment of mastalgia associated with fibrocystic breast condition suggested both efficacy and safety (Ghent et al. 1993). The efficacy of I₂ therapy for treatment of mastalgia was subsequently confirmed in a six month dose ranging study in 111 women (Kessler 2004).

In the above mentioned studies by Ghent and Kessler the dose of I₂ was not associated with increases in incidence, severity, and causality of treatment-emergent adverse events or clinically significant changes in laboratory parameters or vital signs compared to placebo. For instance, in the 6 month study by Kessler headaches were the only adverse event that occurred in a different proportion among the treatment groups; 41% of the placebo group reported headaches as compared to 6.3% of 1.5 mg/day group, 26% of the 3.0 mg/day group, and 12% of the 6.0 mg/day group (p<0.05). No statistically significant change was observed in any of five thyroid function tests (T₃, T₄, T uptake, TSH, and FT₃) for any treatment group, as the mean changes were all within the normal range and considered not clinically significant. No significant shifts either up or down in change from baseline to minimum or maximum were observed. No statistically significant differences among treatment groups were observed for mean change from baseline to minimum except for T₄ (p<0.05).

By and large the literature suggests that the administration of an elevated daily level of iodine under the care of a physician is safe. For instance, over 85 years ago school age children in the Midwest were dosed with 11 mg/d of sodium iodide to determine if this regimen would prevent goiter; control patients did not receive iodine. The iodine treatment group, 2.5 years later, had a 0.2 percent incidence of goiter, whereas the control group had a 22 percent incidence of goiter. Iodine has been used for a wide variety of ailments in the 19^{th} and early part of the 20^{th} centuries, especially for syphilis and chronic lung disease. For many years physicians used potassium iodide in doses starting at 1.5 to more than 10 g/day for weeks at a time to treat bronchial asthma and chronic obstructive pulmonary disease and few side effects are reported. Dermatologists continue to treat inflammatory dermatoses, like nodular vasculitis and pydoderma gangrenosum, with supersaturated potassium iodide, beginning with an iodine dose of 900 mg/day, followed by weekly increases of up to 6 g/day as tolerated. There is no suggestion in the literature that dose is related to adverse events in the targeted population.

In the treatment of the medical conditions identified in this application compliance during the first several months of therapy is important since many drugs with documented efficacy for several of these conditions (e.g. bromocriptine, tamoxifen, danazol) require several menstrual cycles to have an effect. The likelihood of a patient remaining on therapy is expected to be a function of the degree of improvement that a patient experiences during the first several months of treatment. The degree of improvement that will satisfy a patient varies; for instance, studies demonstrate that over 90% of patients are satisfied by a 50% reduction in pain but a 30% reduction may be meaningful in some. Reduction of breast lumps may be extremely important to some women, e.g. women with mothers who developed breast cancer, but insignificant to others. What is clear however, is that the degree of symptom relief experienced within the first several months influences compliance. Consequently, the loading dose of I₂ used to treat the indications identified herein is targeted to provide substantial clinical benefit within the first three months. At the end of the first three months and after the patient's symptoms have been treated, the incidence of adverse events associated with chronic therapy is minimized by lowering the daily dose of I₂ to the minimum necessary to maintain clinical benefit which preferably is no greater than about 50-60% of the loading dose.

The data in this application has allowed us to identify the appropriate range of daily I₂ to use during the initial loading phase of therapy. The data in this application also identify the lowest effective range of I₂ to use for maintaining patients on iodine therapy chronically.

Further, the data in this application also supports the theory that the daily dose of I₂ is correlated with the rate of sub-clinical hypothyroidism. Consequently, establishing the lowest dose of I₂ that maintains clinical improvement during treatment is important. The controlled clinical studies contained in this application demonstrate for the first time that the daily dose of I₂ is an important safety consideration during chronic iodine therapy in the intended population.

### SUMMARY OF THE INVENTION

The method of the present invention involves treatment of medical conditions comprising daily administration of a loading dose of molecular iodine for a period of time adequate to achieve symptomatic clinical relief followed by chronic administration of a daily maintenance dose of molecular iodine at a concentration level substantially below the loading dose.

The method of the present invention further involves administering the loading dose for 2 to 6 months.

In a preferred embodiment of the method of the present invention, the loading dose is administered for 3 months.

In an additional preferred embodiment of the present invention, the loading dose of molecular iodine ranges from 4.5 mg/d to a high of 12 mg/day.

In a further preferred embodiment of the present invention, the chronic dose of molecular iodine ranges from 1.5 mg/d to a high of 3 mg/d.

In a secondary embodiment of the present invention, there is disclosed a method for therapeutically treating medical conditions in female human patients comprising first administering to a patient a daily loading dose of molecular iodine (I₂) for 2 to 6 months at an amount sufficient to remediate symptoms associated with the medical condition followed by daily dosing with a maintenance dose of I₂ after treatment with the loading dose is completed at a concentration level substantially below the loading dose.

The method of the additional embodiment of the present invention involves the daily maintenance dose being lower than the daily loading dose and preferably no greater than 50-60% of the loading dose.

In a further preferred embodiment of the method of the present invention, the maintenance dose is selected to be sufficient to maintain the therapeutic benefit realized by the loading dose.

In an additional preferred embodiment of the method of the present invention, the maintenance dose serves to reduce an incidence of thyroid related findings relative to the loading dose.

### DETAILED DESCRIPTION OF THE INVENTION

This application and the data contained herein are limited to a well defined subset of the general population who do not experience an increased rate of risk of adverse events from daily iodine therapy. Stoddard et al. demonstrated that I₂ alters gene expression in breast cancer cell lines effectively reducing the concentration of estrogen receptor. Experiments in this application confirm this observation. The use of I₂ for treatment of fibrocystic breast disease, endometriosis, ovarian cysts, uterine fibroids and cancer prevention wherein said prevention is based at least in part upon reduction of estrogen receptors in the tissue of interest requires daily dosing of I₂ chronically. The daily dose of I₂ is anticipated to range from a low of about 1.5 mg/d to 12 mg/d. The medical literature indicates that there is no correlation between dose and the frequency of thyroid related adverse events within this concentration range; indeed, the literature suggests that no dose-response exists.

The method of the present invention provides the optimal approach to dosing I₂ which results in an improvement in the medical condition of interest with the lowest possible incidence of thyroid related adverse events. The method of the present invention provides the first clear demonstration that a daily dose of I₂ within the range of 1.5 to 12 mg/d is correlated to thyroid related findings, thereby establishing the lowest dose of I₂ that maintains clinically significant improvements in symptoms during chronic treatment provides the safest course of therapy.

The method of the present invention identifies the optimal approach for dosing I₂ for fibrocystic breast disease, endometriosis, ovarian cysts, uterine fibroids and cancer prevention wherein said prevention is based at least in part upon reduction of estrogen receptors in the tissue of interest.

For convenience, certain terms employed in the specification, examples, and appended claims are defined below.

The term “molecular iodine” as used herein, refers to diatomic iodine which is represented by the chemical symbol I₂, other terms for this species include elemental iodine, iodine metal and simply iodine.

The term “iodide” or “iodide anion” refers to the species which is represented by the chemical symbol I⁻. Common counter-ions for the iodide anion include sodium, potassium, calcium, and the like.

The term “sub-clinical hypothyroidism” refers to a diagnosis by a physician when there are no clinical symptoms of thyroid hormone dysfunction (such as fatigue, cold intolerance, weight gain or memory problems) but a mildly elevated thyroid stimulating hormone (TSH) level and a normal or slightly low level of thyroxin (T₄).

The term “sub-clinical hyperthyroidism” refers to a diagnosis by a physician when there are no clinical symptoms of thyroid hormone dysfunction (such as anxiety, emotional liability, weakness, tremor, palpitations, heat intolerance, increased perspiration, and weight loss) but depressed level of TSH with a normal level of T₄.

The term “overt hypothyroidism” refers to a diagnosis by a physician caused by an imbalance in thyroid hormones. TSH is elevated and both triiodothyronine (T₃) and T₄ are depressed. Clinical symptoms include slowed speech, impaired memory, impaired cognitive function, sensitivity to heat and cold, slow heart rate with ECG changes including low voltage signals, sluggish reflexes, dry puffy skin, anemia, weight gain, constipation, fatigue, anxiety, increased need for sleep, and irritability.

The term “overt hyperthyroidism” refers to a diagnosis by a physician caused by an imbalance in thyroid hormones. TSH is depressed and both triiodothyronine (T₃) and T₄ are elevated. Clinical symptoms include anxiety, emotional liability, weakness, tremor, palpitations, heat intolerance, increased perspiration, and weight loss despite a normal or increased appetite.

The term “clinical relief” or “clinical benefit” refers to (a) a reduction in symptoms associated with the medical condition that satisfies the patient, e.g. pain reduction, or (b) a reduction in an objective medical parameter, e.g. fibrosis, endometrial estrogen receptor concentration, that has medical import.

The term “maintaining therapeutic benefits” refers to the fact that the maintenance dose must insure that the medical benefit derived from the loading dose is not lost.

The term “daily” in reference to a therapeutic regimen refers to administration of a therapeutic every 24 hours for at least 5 days per week and preferably for 7 days each week.

The term “chronic” in reference to a therapeutic regimen refers to a period of time that is at least 3 months of daily duration and can be as long as 30 years in duration.

The term “general population” refers to all individuals in a geographic territory regardless of their thyroid status and without regard to their general health.

The term “intended population” refers to individuals who are euthyroid with no previous history of thyroid disease and excludes individuals with cystic fibrosis, chronic lung disease, the elderly or individuals on lithium therapy.

Many clinicians believe that elevated daily intake of iodine is safe and therapeutic (Miller 2006) for the general public. Indeed, some clinicians support a daily regimen of 50 mg of iodine for the general public and do not believe that this is associated with any adverse thyroid related findings (Abrahams G E, 2004; Abrahams G E, 2005) in the general public. These beliefs have a basis in the scientific literature and current clinical experience. Nevertheless, physicians have identified the subset of the general population that has a greater risk of adverse events from daily oral iodine therapy (Braverman 1994).

Individuals who are intended to be treated by the methods identified in this application should be under the supervision of a physician who can identify individuals at an increased rate of risk of experiencing adverse events from oral iodine therapy; these individuals should not be treated with chronic daily iodine therapy. Therefore, intended population of this application is limited to individuals who are euthyroid with no previous history of thyroid disease. In addition, individuals with cystic fibrosis, chronic lung disease, the elderly or individuals on lithium therapy are excluded since they should not receive daily iodine therapy. The controlled clinical studies contained in this application demonstrate for the first time that the daily dose of I₂ is an important safety consideration during chronic iodine therapy in the intended population.

Sub-clinical hyperthyroidism is not correlated to an increased rate of either overt hyperthyroidism or overt hypothyroidism. On the other hand, it is well established that sub-clinical hypothyroidism is a risk factor for overt hypothyroidism. In fact, if both (1) antibodies to thyroid peroxidase and (2) sub-clinical hypothyroidism are present the risk of subsequently developing overt hypothyroidism in women is at least 50% (Hefland M 2004).

This application contains the first set of controlled data that demonstrate that the daily dose of I₂ is correlated with the rate of sub-clinical hypothyroidism. Consequently, establishing the lowest dose of I₂ that maintains clinically significant improvements in symptoms during treatment is important. The data in the application identifies the appropriate range of daily I₂ to use during the initial loading phase of therapy. The data in this application also identify the lowest effective range of I₂ to use for maintaining patients on iodine therapy chronically which in accordance with the present invention must be at a concentration level below the loading dose and preferably no greater than 50-60% of the loading dose.

### EXAMPLES

### Example 1

Chronic clinical breast pain associated with fibrocystic breast tissue is a chronic condition that lasts an average of about 10 years. The condition is believed to be due to a subtle hormonal imbalance. A limited number of hormonal agents like bromocriptine, taxoxifen, danazol, etc. have demonstrated efficacy in controlled human clinical trials. Patient compliance in the treatment of chronic medical conditions is an issue; this is especially true during initiation of therapy since drugs that are effective against clinical mastalgia usually require at least 2 complete menstrual cycles to have an effect. Consequently, the initial dose of I₂ Of used to treat this category of patient should provide relief within the first several months. The data contained in this example demonstrates that the majority of symptoms relief from I₂ therapy can be accomplished within the first 3 months of therapy provided an adequate dose of I₂ is provided.

A multi-center, randomized, double-blind, placebo-controlled study dosed patients for 6 months with 0.0, 1.5, 3.0 and 6.0 mg I₂ daily. Healthy euthyroid mastalgia patients between the ages of 18 and 50 who were unresponsive to conservative treatments like OTC analgesics were eligible for the study. Subjects needed to identify a history of at least six months of moderate or severe breast pain and document at least 6 days per cycle of moderate or severe pain in a daily dairy (FIG. 1 below). The magnitude of pain reduction increased with dose in a statistically significant manner at both month 3 (p<0.01) and month 6 (p<0.01). The majority of change in the Lewin Overall pain scale occurred by month 3 for the three treatment groups and all of the benefit in the 6.0 mg/d group was observed by month 3, i.e. 45.4% vs. 45.0% respectively. This data demonstrates that the majority of symptoms relief from iodine therapy can be accomplished within the first three months of therapy provided an adequate dose of I₂ is provided.

Mastalgia symptoms associated with fibrocystic breast condition is experienced for an average of about 10 years but can be experienced for over 30 years. Patient compliance with therapy is therefore essential to achieve and maintain clinical benefit. A suitable loading dose of I₂ is able to remediate the symptoms of mastalgia within 2-3 months and therefore provide optimal patient compliance.

The data demonstrates that (1) the 6 mg/d dosing regimen provides greater relief during the first 3 months of therapy than either the 1.5 or 3 mg/d dose and (2) the majority of pain relief with the 6 mg/d dosing regimen is realized within the first 3 months of therapy. The data suggests that 6 mg/d is preferable to either the 1.5 or 3 mg/d dose regimen as a loading dose provided there is no significant difference in adverse event profile between the treatments during the first several months of therapy. Since the study did not find any difference among dosing groups with respect to adverse events or thyroid related clinical chemistry tests during the first 3 months of treatment the 6 mg/d is considered the preferred minimum dose for loading therapy.

### Example 2

Female patients already receiving aqueous I₂ therapy for fibrocystic breast condition were enrolled in study that ran for 36 months. Patients in this study were dosed at I₂ levels that ranged from 3 to 12 mg/d. A total of 923 patients were enrolled; over 700 patients remained on drug for at least 2 years. The ages of participants ranged from 17 to 78 years with an average age of 47.4 years.

Variations in treatment emergent thyroid status were evaluated based on TSH and T₄ values (see Table 1). Patients were categorized sub-clinical hyperthyroid if TSH was below the normal range (0.35-5.00 mU/L) and T₄ values were within the normal range (60-155 nmol/L); patients that exhibited depressed TSH and elevated T₄ values were scored as overtly hyperthyroid. Patients were categorized sub-clinical hypothyroid if TSH was elevated and T₄ values were normal; patients with depressed T₄ values were considered overtly hypothyroid. Overt hypothyroidism occurred in 0.2% of the patients in this study which is comparable to the estimated prevalence (2%) of overt hypothyroidism in adult women. Overt hypothyroidism occurred in 0.5% of the patients in study which is also comparable to the estimated prevalence (2%) of overt hyperthyroidism in adult women. These data confirm prior published clinical reports from clinicians indicating that overt thyroid disease is not associated with iodine therapy.

TABLE 1
Thyroid Emergent Findings in Iodine Adapted Patients Exposed
to Chronic Daily Iodine Therapy

| | | | | Hypothyroidism | | | | | |
|---|---|---|---|---|---|---|---|---|---|
| | Patients | Hyperthyroidism | | | | | Overt | | |
| | with | | Overt | Subclinical | | | ↑TSH | ↑TSH | ↑TSH |
| Dose | Lab | Subclinical | ↓TSH | ↑TSH | ↑TSH | ↑TSH | (5-10) | (10-20) | (>20) |
| I₂ (mg/d) | Values | ↓TSH | ↑T₄ | (5-10) | (10-20) | (>20) | ↓T₄ | ↓T₄ | ↓T₄ |
| 3.0 | 300 | 16 (5.3) | 1 (0.3) | 27 (9.0) | 10 (3.3) | 2 (0.7) | 2 (0.4) | 0 (0.0) | 0 (0.0) |
| 6.0 | 490 | 28 (5.7) | 1 (0.2) | 70 (14) | 10 (2.0) | 1 (0.2) | 0 (0.0) | 2 (0.4) | 2 (0.4) |
| 9.0 | 16 | 0 (0.0) | 0 (0.0) | 0 (0.0) | 1 (6.3) | 1 (6.3) | 0 (0.0) | 0 (0.0) | 0 (0.0) |
| 12.0 | 4 | 1 (25) | 0 (0.0) | 0 (0.0) | 0 (0.0) | 0 (0.0) | 0 (0.0) | 0 (0.0) | 0 (0.0) |

The overall incidence of treatment emergent sub-clinical hyper- or hypothyroidism was 20.6%. This incidence is higher than that reported in earlier studies. The proportion of patients with findings of sub-clinical hypothyroidism was 13% and 16.5% in the 3 and 6 mg dose group. If we compare the rate of all sub-clinical conditions (hyperthyroidism and hyperthyroidism) we see that the rate for the 3 and 6 mg dose groups are 18.3 and 22.2% respectively. A comparison of the ratio of patients that experienced mild sub-clinical hypothyroidism (TSH 5-10) in the 3 (27/300) and 6 (70/490) mg/d dose groups demonstrates a statistically significant difference (p=0.0334; Fisher's Exact test). This controlled data demonstrates that a chronic daily regimen of iodine in the concentration range between 3 and 6 mg/d is associated with an increase in sub-clinical thyroid conditions.

Unlike sub-clinical hyperthyroidism, sub-clinical hypothyroidism has been shown to be a strong risk factor for future development of overt thyroid disease. This is particularly true for women. For instance, a 50-year-old woman with normal TSH and negative thyroid peroxidase antibodies has a risk of only 4% of developing overt thyroid disease between the ages of 30 and 50. A mildly elevated TSH of 6 mU/L with positive anti-thyroid antibodies increased this risk to 57% over the same time frame. A reduction in sub-clinical hypothyroidism in women between the ages of 30 and 50 therefore reduces the incidence of overt thyroid disease. Consequently, the 3 mg/d day dose is preferred to the 6 mg/d dose for use as the maintenance dose of 12.

### Example 3

A second I₂ safety study was conducted in women not previously exposed to iodine therapy (iodine naïve). This study enrolled 389 patients: placebo (n=67); 0.3 mg/d (n=66); 2.0 mg/d (n=63); 3.0 mg/d (n=66); 6.0 mg/d (n=65); and 9.0 mg/d (n=62). Thyroid functions laboratory values were available for 365 patients. The age of the female participants ranged from 20 to 55; the average age was 42.4 years. Variations in treatment emergent thyroid status were evaluated based on TSH and T₄ values (see Table 2). Patients were categorized sub-clinical hyperthyroid if TSH was below the normal range (0.35-5.00 mU/L) and T₄ values were within the normal range (60-155 nmol/L); patients that exhibited depressed TSH and elevated T₄ values were scored as overtly hyperthyroid. Patients were categorized sub-clinical hypothyroid if TSH was elevated and T₄ values were normal; patients with depressed T₄ values were considered overtly hypothyroid. Overt hypothyroidism occurred in 1.3% of the patients which is comparable to the estimated prevalence (2%) of overt hypothyroidism in adult women. Overt hypothyroidism occurred in 1.6% of the patients is also comparable to the estimated prevalence (2%) of overt hyperthyroidism in adult women.

The incidence of abnormal thyroid function in iodine naïve patients receiving therapy did not differ between placebo and the treatment groups. Fisher's exact test did not reach significance for comparisons of sub-clinical hyperthyroidism, overt hyperthyroidism, sub-clinical hypothyroidism, overt hypothyroidism, or sub-clinical hyper- or hypothyroidism. Sub-clinical hyperthyroidism is not correlated to an increased rate of either overt hyperthyroidism or overt hypothyroidism. On the other hand, it is well established that sub-clinical hypothyroidism is a risk factor for overt hypothyroidism. In fact, as stated in Example 2, antibodies to thyroid peroxidase combined with sub-clinical hypothyroidism are established risk factors for overt thyroid disease in women between the age of 30 and 50. Therefore the key question is whether or not dose is related to the frequency of sub-clinical hypothyroidism. Kendall's rank correlation was used to evaluate the relationship between dose and frequency of sub-clinical hypothyroidism. Kendall's rank correlation (exact) yielded a two sided p value of 0.0167 indicating that the daily dose of I₂ is correlated to the frequency of sub-clinical hypothyroidism. Cuzik's trend test also demonstrated a statistically significant relationship (p value of less then 0.01) between dose and rate of sub-clinical hypothyroidism.

TABLE 2
Thyroid Emergent Findings in Iodine Naive Patients
Exposed to Chronic Daily Iodine Therapy

| | | | | Hypothyroidism | | | | | |
|---|---|---|---|---|---|---|---|---|---|
| | Patients | Hyperthyroidism | | | | | Overt | | |
| | with | Sub- | Overt | Sub-clinical | | | ↑TSH | ↑TSH | ↑TSH |
| I₂ | Lab | clinical | ↓TSH | ↑TSH | ↑TSH | ↑TSH | (5-10) | (10-20) | (>20) |
| (mg/d) | Values | ↓TSH | ↑T₄ | (5-10) | (10-20) | (>20) | ↓T₄ | ↓T₄ | ↓T₄ |
| 0 | 63 | 0 (0.0) | 0 (0.0) | 3 (4.8) | 0 (0.0) | 0 (0.0) | 0 (0.0) | 0 (0.0) | 0 (0.0) |
| .03 | 65 | 1 (1.5) | 0 (0.0) | 3 (4.6) | 0 (0.0) | 0 (0.0) | 0 (0.0) | 0 (0.0) | 0 (0.0) |
| 2.0 | 58 | 2 (3.4) | 0 (0.0) | 3 (5.2) | 2 (3.4) | 0 (0.0) | 1 (1.7) | 0 (0.0) | 0 (0.0) |
| 3.0 | 62 | 0 (0.0) | 2 (3.2) | 5 (8.1) | 0 (0.0) | 0 (0.0) | 0 (0.0) | 0 (0.0) | 0 (0.0) |
| 6.0 | 60 | 2 (3.3) | 0 (0.0) | 6 (10) | 0 (0.0) | 0 (0.0) | 0 (0.0) | 0 (0.0) | 1 (1.7) |
| 9.0 | 57 | 2 (3.5) | 2 (3.5) | 8 (14) | 0 (0.0) | 0 (0.0) | 2 (3.5) | 0 (0.0) | 1 (1.8) |

### Example 4

A patient (#647-initials NM) diagnosed with appendicitis underwent surgery during which the appendix was found to be normal. The post surgical diagnosis was ruptured follicular cyst of the right ovary. After the surgical procedure the patient continued to experience lower right quadrant abdominal pain. An ultrasound subsequently exhibited a follicular cyst 3 cm in diameter in the left ovary. The patient was started on I₂ therapy at a dose of 6.0 mg/d. The patient was seen again 3 months later (December 3 to March 16). After three months of I₂ therapy the patient was free of pain which her mother regarded as unique.

The patient subsequently stopped I₂ therapy and approximately 3 years later symptoms of abdominal and breast pain were present. The patient was started again on daily I₂ therapy at a dose of 6.0 mg/d. An ultrasound was performed and small follicular cysts in each ovary was noted. Within a month of reinitiating I₂ therapy at a dose of 6.0 mg/d symptoms of pain were no longer present. An ultrasound was performed on the patient approximately 4 months later (November 2 to March 24) and the ovaries appeared normal. The patient was continued on daily iodine therapy at a concentration of 3.0 mg/d. The patient was seen 9 and 18 months later and she remained asymptomatic.

The safe upper limit (UL) of daily dietary intake of iodine for the general public has been identified as 1.1 mg/d. Some physicians advocate for daily oral administration of iodine levels that are 5 to 50 times higher than the UL (Miller 2006). These advocates point to the fact that daily iodine therapy at levels ranging from 10 to 50 times the UL were prescribed by physicians for much of the 20^{th} century and no apparent increases in adverse events were observed. Epidemiologic studies of groups of individuals living in specific areas with elevated average dietary iodine intake have drawn an association between daily iodine intake and the incidence of sub-clinical hypothyroidism in the general public. However, there are no controlled studies that confirm such relationships in the general public.

It is known that certain categories of individuals are more likely to experience thyroid related adverse events when suddenly exposed to daily iodine levels above the UL (Braverman 1994). Most individuals in the general population are known to tolerate daily iodine administration without associated side effects. This application and the data contained herein is limited to this well defined subset of the general population who do not experience an increased rate of risk of adverse events from daily iodine therapy. Consequently, this application is limited to euthyroid women with substantially or essentially no previous history of thyroid disease and excludes individuals with cystic fibrosis, chronic lung disease, the elderly or individuals on lithium therapy.

### Example 5

A total of 24 nulliparous and nonpregnant female Sprague-Dawley rats were received from Charles River Laboratories, Kingston, N.Y. The animals were 10 weeks of age and were examined for signs of disease or injury upon receipt. The animals were housed in stainless steel cages and fed LabDiet™ Certified Rodent Chow 5002 (PMI Feeds, Inc., a subsidiary of Purina Mills, Inc.) ad libitum and were provided fresh filtered tap water ad libitum. Environmental controls were set to maintain a temperature of 18 to 26° C. and a relative humidity of 30 to 70%. A 12-hour light/dark cycle was employed and the room underwent a minimum of ten fresh air changes/hour.

The animals were allowed to acclimate to laboratory conditions for 2 weeks. No abnormal findings excluding animals from study were noted. The animals were released from conditioning following an examination. Each animal was identified with a unique number that was indicated by ear punch. Cage labels, color-coded by group, identified each cage. The animals were maintained at laboratory conditions for an additional 2 weeks and then randomized into treatment groups by weight. At randomization, the weight variation of animals did not exceed ±20% of the mean weight.

Tablets of IoGen, a registered trademark of Symbollon Pharmaceuticals Inc. of Framingham Mass. USA were dissolved in distilled water. IoGen tablets are compositions of iodide and iodate in a pharmaceutically acceptable carrier which will form molecular iodine “I₂” once in contact with the gastric fluid in the stomach via a diffusion controlled oxidation reduction reaction as taught in U.S. Pat. No. 5,885,592 the disclosure of which is incorporated herein by reference. For this example the IoGen tablets weighted 400 mg and contained the equivalent of 6 mg of I₂ in the form of iodide and iodate. The dissolved IoGen tables were used to provide defined daily doses of I₂ to rats according to Table 1 below.

TABLE 1
Daily I₂ Dosing Schedule for Female Rats

| | Dose Level | | Dose | |
|---|---|---|---|---|
| | (mg | Concentration^{a} | Volume | |
| Group | I₂/kg/day) | (mg/mL) | mL/kg | Number |
| 1 | 0 | 0 | 10 | 6 |
| 2 | .06 | .006 | 10 | 6 |
| 3 | 0.2 | 0.02 | 10 | 6 |
| 4 | 2 | 0.204 | 10 | 6 |

^{a}Equivalent concentration of I₂ from IoGen tablet in the dosing solution.

After randomization vaginal smears were taken from rats using a cotton swab; the swab was inserted into the vagina and rotated 360° in a clockwise direction, then the swab was smeared onto a glass slide and the smear was fixed in 3.7% formaldehyde with phosphate buffered saline (PBS) for 15 minutes. The slides were washed in PBS for 5 minutes and subsequently immersed in cold methanol and acetone (both at −10° C.) for 3 minutes. After a final rinse in cold PBS for 5 minutes the slides were stored at −20° C. in a solution of PBS-glycerol.

Rats were then dosed daily for the next 2 months according to the schedule in Table 1. There were no statistically significant differences between placebo (Group 1) and any of the treated groups with respect to daily feed consumption or weight. After 2 months of daily dosing vaginal swabs were taken again and slides were prepared as on day 1 of the dosing regimen.

Immunocytochemical (IC) detection of estrogen receptor (hereafter “ER”) status was accomplished using mouse monoclonal antibody 6F11 from Vector Laboratories (Burlingame, Calif.). A gentle stream of 10 mM citrate buffer (pH 6.0) was passed over each slide for 45 minutes and then a 1/25 dilution of the antibody was incubated with slides at room temperature for 30 minutes. Slides were washed and the Vectastain ABC Kit for Mouse IgG (Vector Laboratories; Burlingame, Calif.) was used to visualize ER. The chromophone 3,3′ diaminobenzidine and peroxidase were used to stain areas of the slides that contained ER. The intensity of staining was scored semi quantitatively on a scale of +1 to +3. Each dosing group contained 6 animals. The mean and standard deviation for IC of ER at the beginning and end of dosing is shown in the table below.

Immunocytochemical Detection of Estrogen Receptor before and
after 2 Months of Daily I₂ Treatment in Sprague-Dawley Rat

Scores (mean ± SD)

| Group | Baseline | End |
|---|---|---|
| 1 | 2.167 ± 0.983 | 2.000 ± 0.632 |
| 2 | 2.000 ± 0.632 | 1.167 ± 0.752 |
| 3 | 2.167 ± 0.753 | 1.167 ± 0.408 |
| 4 | 2.333 ± 0.516 | 0.833 ± 0.408 |

No statistically significant difference in ER concentration was found at baseline between placebo and any of the other experimental treatments. A comparison of the different dosing groups to placebo with the Kruskal-Wallis test generated p values of 0.974, 0.999 and 0.999 versus the 0.02, 0.06 and 2.0 mg/kg dosing groups. A statistically significant difference in the change from baseline to end was observed between placebo and all rats that received I₂ (unpaired t-test; p=0.015). Cuzik's trend test was used to test for trend across the 3 dosing groups and a statistically significance was reached (p<0.001). The data indicates that daily I₂ treatment is correlated with down regulation of ER in the endometrium.

Down regulation of ER has clinical significance. Many women who are at an increased risk of breast cancer are placed on the drug tamoxifen. Tamoxifen and several of its metabolites act as estrogen antagonists by binding to ER. It appears that I₂ reduces the concentration of ER which could account, in part, for the antiproliferative properties reported in the literature. Women who take tamoxifen chronically to reduce their risk of breast cancer are known to experience in increased risk of ovarian cancer.

I claim:

1. A method for treating a medical condition in a human requiring chronic therapy selected from the group of medical conditions consisting of: fibrocystic breast disease, endometriosis, ovarian cysts, uterine fibroids and including the treatment of women considered to be at risk to breast and/or ovarian cancer without causing an overt thyroid disease comprising the steps of: chronic administration of a loading dose of molecular iodine for a first fixed period of time at a concentration level which will provide clinical relief sufficient to remediate symptoms associated with the medical condition followed by the chronic administration of a daily maintenance dose of molecular iodine for a second fixed period of time with the concentration of the maintenance dose being substantially less than the concentration of the loading dose.

2. The method of claim 1 wherein the concentration of the maintenance dose is no greater than about 50-60% of the loading dose.

3. The method of claim 2, wherein the loading dose of molecular iodine ranges from between 4.5 mg/d to a high of 12 mg/day.

4. The method of claim 3 wherein the loading dose is administered for 2 to 6 months.

5. The method of claim 4, wherein the loading dose is administered for 3 months.

6. The method of claim 4 wherein the chronic loading dose of molecular iodine ranges from between 1.5 mg/d to a high of 3 mg/d.

7. A method for therapeutically treating iodine deficiency conditions in female human patients comprising first administering to a patient a daily loading dose of molecular iodine (I₂) for 2 to 6 months at an amount sufficient to remediate symptoms associated with the iodine deficiency disease followed by daily dosing with a maintenance dose of I₂ after treatment with the loading dose is completed wherein the daily maintenance dose is lower in concentration than the concentration of the loading dose.

8. The method of claim 7 wherein the maintenance dose is selected to be sufficient to maintain the therapeutic benefit realized by the loading dose.

9. The method of claim 8, wherein the maintenance dose serves to reduce an incidence of thyroid related findings relative to the loading dose.

10. The method of claim 9 wherein the maintenance dose is selected to be no greater than about 50-60% of the loading dose.

11. The method of claim 9, where in the loading dose of molecular iodine ranges from 4.5 mg/d to a high of I₂ mg/day.

12. The method of claim 11, wherein the chronic dose of molecular iodine ranges from 1.5 mg/d to a high of 3 mg/d.

13. A method for controlling the level of estrogen receptors in women considered at risk to breast and/or ovarian cancer consisting of administering a loading dose of molecular iodine for an interval of time not to exceed 6 months at a concentration not to exceed 12 mg/day followed by a daily maintenance dose of molecular iodine for a second interval of time with the concentration of the maintenance dosage being less than the concentration of the loading dose.

14. The method of claim 13 wherein the maintenance dose is selected to be no greater than about 50-60% of the loading dose.
